(19) United States
(12) Patent Application Publication
(10) Pub. No.: US 2007/0059767 A1
(43) Pub. Date: Mar. 15, 2007

(54) Method of identifying N-terminal proBNP

(75) Inventors: Johann Karl, Peissenberg (DE); Helmut Lill, Wielenbach (DE); Peter Stahl, Bernried (DE); Kerstin Krueger, Muenchen (DE); Anneliese Borgya, Seeshaupt (DE); Andreas Gallusser, Penzberg (DE)

Correspondence Address: BRINKS HOFER GILSON & LIONE, P.O. BOX 10395, CHICAGO, IL, 60610, US

(73) Assignee: Roche Diagnostics GmbH

(21) Appl. No.: 11/500,555

(22) Filed: Aug. 7, 2006

Related U.S. Application Data
(63) Continuation of application No. 09/890,442, filed on Jan. 31, 2002.

(30) Foreign Application Priority Data
Jan. 29, 1999 (DE) 19903489.3
Mar. 12, 1999 (DE) 19911044.1

(51) Int. Cl. G01N 33/53; C12N 5/06; C07K 16/22

(52) U.S. Cl. 435/7.1; 435/326; 530/388.25

(57) ABSTRACT

The invention relates to a method of identifying N-terminal proBNP in a sample with at least two antibodies that detect different epitopes of the N-terminal proBNP. The method is used to differentiate or classify samples of healthy individuals and samples of patients of NYHA classes I to IV. The invention further relates to recombinant N-terminal proBNP, its use as standard in a method of identifying N-terminal proBNP, to antibodies that detect recombinant N-terminal proBNP and to their production.

The present invention concerns a method of identifying N-terminal proBNP in a sample with at least two antibodies that detect different epitopes of the N-terminal pro BNP. The method is used to differentiate or classify samples of healthy individuals and samples of patients of NYHA classes I to IV. The invention further concerns recombinant N-terminal proBNP, its use as standard in a method of identifying N-terminal proBNP, antibodies that detect recombinant N-terminal proBNP and their production.

Heart failure is a widespread phenomenon especially in the western world. According to the Roche medical dictionary (1993, Urban & Schwarzenberg) heart failure is the acute or chronic inability of the heart to generate the blood flow required for the metabolism during exercise or even at rest or to assure the venous reflux (backward and forward failure). Thus the pump function of the heart is weak. The causes of heart failure are very complex. Among others, inflammatory and degenerative modifications of the cardiac muscle, coronary perfusion disorder, coronary infarction and injuries are mentioned here. This leads to modifications of the peripheral bloodstream, disorder of the breathing, renal function and electrolyte metabolism (oedema) and to a reduced performance of the muscular system of the skeleton.

According to the New York Heart Association (NYHA) heart failure is divided into the following NYHA classes using physical tests after effort: I means completely free from pain after normal physical effort, II means low limitation of the physical toughness, III means strong limitation of the physical toughness, IV means that with each physical activity the insufficiency symptoms increase which most of the time also exist at rest.

For an effective medicament treatment of heart failure by means of glycosides, vasodilators, ACE inhibitors and/or B-blockers it is first of all necessary to exactly diagnose the heart failure and to classify it if possible according to the severity degree and to additionally monitor the course of the treatment.

According to the state of the art some serum markers for an early diagnosis of heart failure as for example ANP (N-terminal atrial natriuretic peptide hormone) and pro ANP, CNP (C-natriuretic peptide), adrenomedullin, neuropeptide Y, endotheline and BNP (brain natriuretic peptide) are discussed. ANP and proANP are generally suitable as markers for the diagnosis of heart failure; they are however not very stable or only have a short half life in the blood which represents an impediment to diagnostic measurements (Clin. Sci. 95(3) (1998), 235-239; Cleland et al., Heart 75 (1996), 410-413).

A frequently cited and meaningful marker is BNP (brain natriuretic peptide). Originally, BNP was identified in the brain of pigs. It is a cardiac hormone which structurally and functionally resembles to ANP (atrial natriuretic peptide) (Sudoh et al., Nature 332 (1988), 78-81). Human BNP consisting of 32 amino acids is mainly secreted by the heart ventricles and circulates in the human blood plasma. The use of BNP as a diagnostic marker is for example known from EP-A-0 542 255. BNP has an intramolecular disulfide bridge and is not very stable as an analyte presumably due to its physiological function as a hormone that must be broken down quickly. Therefore, its use as a diagnostic marker is only limited (Masuta et al., Clin. Chem. Vol. 44 No. 6 Supplement A (1998), 130; Tsuji et al., Clin. Chem. 40 (1994), 672).

The precursor molecule of BNP, i.e. proBNP consists of 108 amino acids, of which the aforementioned 32 C-terminal amino acids (77-108) called BNP develop the real hormonal effect. The N-terminal amino acids 1-76 released from the precursor are called N-terminal proBNP. Besides BNP (77-108) N-terminal proBNP also circulates in the plasma as well as further breakdown products (1-76) (Hunt et al., Biochem. Biophys. Res. Com. 214 (1995), 1175-1183) so that N-terminal proBNP is also relevant as a marker of heart-failure. Whether the precursor molecule proBNP, also occurs in the plasma is not completely resolved. It is however described (Hunt et al., Peptides, Vol. 18, No. 10 (1997), 1475-1481) that a low release of proBNP (1-108) in the plasma is detectable but that due to the very quick partial breakdown at the N-terminal end some amino acids are absent. This molecule is called High Molecular Weight BNP in the literature.

WO 93/24531 (U.S. Pat. No. 5,786,163) describes an immunological method of identifying N-terminal proBNP and the antibodies used for it. To obtain these antibodies single synthetically produced peptides from the sequence of N-terminal proBNP are used here. The production of antibodies by means of peptide immunization is possible in principle but the affinity regarding the whole molecule generally is too low to reach the necessary sensitivity in a test procedure. In addition, there is a danger that when using peptides the antibodies obtained can for example identify the C-terminus of the peptide and can therefore only bind to this fragment of the whole molecule. From this results that these antibodies cannot bind to the whole molecule or only to a low extent. In WO 93/24531 polyclonal antibodies against one single peptide derived from the N-terminal proBNP are produced. It is shown that the antibodies produced bind to the immunization peptide (amino acids 47-64) in the competitive test format. It is however not shown that the antibodies are able to bind to native N-terminal proBNP as a whole molecule in a sample. Additionally, the sandwich test described in WO 93/24531 in a sample cannot be performed as described since there was no appropriate standard material and no antibodies against two different epitopes.

A further problem in the state of the art is the test sensitivity. With the competitive test performed in WO 93/24531 where the peptide 47-64 competes in a labelled form as a tracer with a sample or the unlabelled peptide standard 47-64 to bind to polyclonal antibodies from rabbit serum only a very moderate competition is reached after 48 hours of incubation from which can only be derived a low detection limit of approx. 250 fmol/ml. This is neither sufficient for the differentiation of healthy individuals and patients suffering from heart failure nor for a differentiated classification of patient samples into the severity degrees of heart failure. In addition, the long incubation times of the competitive test are not acceptable for routine measurements of the samples in automated laboratories.

Hunt et al. (Clinical Endocrinology 47 (1991), 287-296) also describes a competitive test for the detection of N-terminal proBNP. For this a complex extraction of the plasma sample is necessary before the measurement; this may lead to the destruction of the analyte and error measurements. The antiserum used is produced analogously to WO 93/24531 by immunization with a synthetic peptide. Hunt et al. produces the antiserum by immunization with the N-terminal proBNP amino acids 1-13 and the peptide of amino acids 1-21 is used as a standard. For this test long incubation times are necessary too. After an incubation of 24 hours a lower detection limit of 1.3 fmol/ml is reached.

Thus, there is no state of the art method to detect N-terminal proBNP which enables a reliable, sensitive detection of native N-terminal proBNP with short incubation periods.

It was therefore an object to provide a method of identifying N-terminal proBNP in a sample avoiding as much as possible the aforementioned disadvantages of the state of the art. In particular a high test sensitivity should be reached to allow a differentiation of the patient samples of healthy individuals and patients of the NYHA classes I to IV.

This object is obtained with the method of identifying N-terminal proBNP in a sample which is explained in more detail in the claims. The method is characterized in that at least two antibodies detecting different epitopes of the N-terminal proBNP are used.

What is important in the method according to the invention is that native N-terminal proBNP is detected in a sample. This means that the antibodies must be able to identify and specifically bind to the intact molecule and possibly occurring uncleaved proBNP (1-108) and if possible also to partially proteolytically digested fragments in a sample. For the method at least two different antibodies are used which bind to different epitopes of the N-terminal proBNP. The epitopes can be linear or so-called conformation epitopes. Preferably the epitopes are localized in a manner enabling both antibodies to bind at the same time and not to be too far away from each other.

Since the method according to the invention does not allow to differentiate between N-terminal proBNP, proBNP and parent peptides (breakdown products) NT-proBNP means in the following all peptides identified in the test procedure, in particular the known N-terminal proBNP (1-76).

According to the invention the term “epitope” means the binding site on an immunological binding partner such as an antigen to which an antibody binds specifically. Usually an epitope is clearly defined by 6 to 8 amino acids. According to the invention the binding partner corresponds to the N-terminal proBNP or a partial sequence thereof. The epitope to which the antibody binds constitutes a partial region on the binding partner. The epitope can be present in a linear form or as a conformation epitope.

By means of the two antibodies with differing specificities it is possible to perform a quicker method of identifying the analyte instead of the long competitive test procedure of the state of the art. The detection method according to the invention can be performed by means of a homogeneous or heterogeneous test procedure. Preferably the heterogeneous test procedure is used and particularly preferably the sandwich procedure known to the expert.

Preferably, such a method of determination of the N-terminal proBNP is performed according to the following steps:
a) Mixing of the sample with the first N-terminal proBNP-specific antibody carrying a group suitable for binding to a solid phase or mixing with the first N-terminal proBNP-specific antibody which has already bound to a solid phase
b) Mixing of this solution with the second antibody identifying an epitope of NT-proBNP differing from that of the first antibody and carrying a label.
c) Binding of the immune complex to a solid phase which can already be present in step a)
d) Separation of the solid phase from the liquid phase
e) Detection of the label in one or both phases.

In a quantitative determination the same measurement is carried out with a defined amount of N-terminal proBNP as a standard and after the determination of the sample step f) is performed, i.e. the comparison of the measuring values of the standard with that of the sample, and then the quantification takes place.

The term “antibody” means—according to the invention—mono- or polyclonal, chimerical or humanized or other antibodies obtainable by genetically engineered modifications as well as all fragments known to the expert such as F(ab′)₂, Fab′ or Fab fragments. Only the immunological specific binding capacity for N-terminal proBNP must be guaranteed.

The first antibody specific for N-terminal proBNP can be bound directly to the solid phase or indirectly via a specific binding system. The direct binding of this antibody to the solid phase follows methods known to the expert, for example in an adsorptive way. If the binding is indirect via a specific binding system the first antibody is a conjugate consisting of an antibody against N-terminal proBNP and a reaction partner of a specific binding system. A specific binding system means here two partners which can react specifically with each other. The binding capacity can be based on an immunological reaction or on a different specific reaction. Preferably, a combination of biotin and avidin or biotin and streptavidin is used as a specific binding system. Further preferred combinations are biotin and antibiotin, hapten and anti-hapten, Fc-fragment of an antibody and antibodies against this Fc fragment or carbohydrate and lectin. One of the reaction partners of the specific binding system is then part of the conjugate.

The other reaction partner of the first binding partner in the specific binding system is a layer of the solid phase. Streptavidin or avidin are used preferably. The binding of the other reaction partner of the specific binding system to an insoluble carrier material can be performed according to the usual methods known to the expert. Here a covalent as well as an adsorptive binding is suitable.

As a solid phase test tubes or microtiter plates made of polystyrene or similar plastics are suitable which are coated at their inner surface with a reaction partner of the specific binding system. Further substances that are suitable and particularly preferred are particle substances such as latex particles, magnetic particles, molecular sieve materials, glass corpuscles, plastic tubes and others. Porous, stratiform carriers such as paper or nitrocellulose can also be used as carriers. Magnetic beads coated with the corresponding binding partner of the specific binding system described above are used particularly preferably. After completion of the test reaction these microparticles can be separated from the liquid phase for the procedure of the detection reaction for example by filtration, centrifugation or in the case of the magnetic particles via a magnet.

The second specific antibody identifies a different epitope of the N-terminal proBNP compared to that of the first antibody. The distance of the two epitopes on the molecule must be large enough so that the simultaneous binding of the antibodies to the N-terminal proBNP is possible without reservation; if not, no sandwich complex can be built.

The detection of the specific binding reactions between the antibodies against N-terminal proBNP and N-terminal proBNP can be performed in different ways. Generally, the second antibody is labelled. Usual labels are chromogens, fluorophores, substances suitable for chemi- or electrochemiluminescence, radioisotopes, haptens, enzyme markers or substances capable of building a specific binding couple such as biotin/streptavidin. The immune complex is then detected by means of the signal emitted by the label; The second antibody can for example be labelled with the hapten digoxigenin. This hapten is again bound by a further digoxigenin-specific antibody. This antibody specific for digoxigenin is itself labelled by an enzyme as e.g. peroxidase. The final detection is then carried out by means of a change in the colour or extinction occurring when a particular substrate is added to the peroxidase.

All biological liquids known to the expert can be used as samples for the procedure of the method of identifying N-terminal proBNP. The samples preferred are body liquids like whole blood, blood serum, blood plasma, urine or saliva. The use of blood serum and plasma is particularly preferred.

Besides the so-called wet tests with test reagents in a liquid phase all usual dry test formats suitable for the detection of antigens, haptens, peptides, proteins, antibodies etc. can be used too. These dry tests or test strips as for instance described in EP-A-0 186 799 combine all test components on one single carrier—except the sample to be analyzed. The detection reaction begins when the test strip gets into contact with the liquid sample.

The method according to the invention is characterized in that the lower detection limit for N-terminal proBNP is less than 1 fmol/ml (corresponds to 1 pmol/l). The high sensitivity of <1 fmol/ml according to the invention is reached without long incubation periods. The total period of a microtiter test is less than 2 hours, preferably about 15 minutes with more sensitive detection methods like electrochemiluminescence. An upper limit regarding the concentration to be detected does practically not exist for this detection method. The technological upper limit is generally given by the measuring method used. The method principally also detects very high concentrations of N-terminal proBNP.

A further advantage of the method according to the invention is the good differentiation of the samples of patients with and without heart failure by means of the measuring values obtained. The detection method is so sensitive that even a differentiation of individuals without a coronary disease and patients suffering from a mild or only a slow onset heart failure of the NYHA classes I and II. Such an early establishment of a beginning heart failure can influence the decision to begin an early treatment with drugs and thus clearly prolong the survival rate of the patient.

Another subject matter of the invention is recombinantly produced N-terminal proBNP. N-terminal proBNP is the N-terminal part consisting of the amino acids 1-76 and released from the precursor molecule proBNP consisting of 108 amino acids.

N-terminal proBNP also embraces parts thereof which may occur in the blood due to breakdown reactions of this molecule.

No recombinant N-terminal proBNP is hitherto known in the state of the art since its production is not easily possible due to the short amino acid sequence. The chemical synthesis of a peptide of more than 30 amino acids is due to the occurring error sequences and the strongly decreasing yield per synthetic cycle no alternative compared to the recombinant production of a host organism.

For a diagnostic detection method a standard or control material is however always necessary to determine the analyte quantitatively on the one hand and to check the functional capability of the test on the other hand. If a quantification is desired a defined quantitative calibration must be performed using a standard series. Such a calibration is however only useful in the case the material used as standard shows the same or a similar behavior in the immunological test with respect to the analyte. It is important that the standard has a sufficient structural and in particular an immunological similarity to the analyte so that the binding of the standard to the detection antibody resembles to that of the native molecule in the sample.

Such a standard material for a method of detection of N-terminal proBNP is not provided by the state of the art. Only short synthetic peptides are described. According to the invention it is now for the first time possible to produce a DNA sequence coding for N-terminal proBNP with the aid of genetic synthesis and to reach a recombinant expression of the N-terminal proBNP in E. coli. Example 1 explains the single steps to follow.

A further subject matter of the invention is therefore the use of recombinant N-terminal proBNP as a standard in a method of identifying N-terminal proBNP in a sample by means of at least two antibodies recognizing different epitopes of the N-terminal proBNP.

For reasons of immunization only synthetic, short peptides derived from N-terminal proBNP have been used in the state of the art. The disadvantage of peptide immunizations is that most of the time only very low-affine antibodies are obtained or the antibodies obtained only react with linear epitopes and the native-fold antigen cannot be bound in the sample (see example 3).

Therefore it is important to use for the production of antibodies an immunogen with a sufficient similarity to the analyte to be detected. Only by this way it can be guaranteed that the antibody binds with a high affinity to the native analyte in the sample.

A subject matter of the invention is therefore also the use of recombinant N-terminal proBNP as an immunogen for the production of antibodies against N-terminal proBNP.

A further subject matter of the invention are antibodies against recombinant N-terminal proBNP. The definition of the term antibody corresponds to the definition given in the paragraphs concerning the test procedure. Preferably, the antibodies according to the invention specifically identify epitopes in the N-terminal part of the 76-amino acid large N-terminal proBNP, preferably in the amino acid region from 10 to 66, particularly preferred in the region 10 to 50 or 10 to 38. A useful localization of the epitopes identified by the antibodies is reached when even N-terminal proBNP which is at its ends already proteolytically digested in the sample contains these epitopes. The stability of the analyte in the sample is thus of a more or less secondary importance. The epitopes in the preferred regions of the N-terminal proBNP can occur in a linear form or as conformation epitopes. A preferred subject matter of the invention are therefore monoclonal antibodies produced by the cell lines MAB M 10.1.1 and MAB M 13.4.14, deposited and received on the 26^{th} of Jan. 1999 with the DSMZ (German collection of microorganisms and cell cultures) GmbH, Braunschweig, Germany. The antibodies produced by these two cell lines are IgG-type antibodies. The cell lines M 10.1.11 and M. 13.4.14 are also a subject matter of the invention.

A further subject matter of the invention are antibodies which are like those of the cell lines M 10.1.11 and M 13.4.14 produced in an equivalent way and suitable for specifically binding to N-terminal proBNP. The expression “antibodies produced in an equivalent way” means that the antibodies are obtained by immunization with recombinant N-terminal proBNP.

A subject matter of the invention are also methods for the production of antibodies specifically binding to N-terminal proBNP.

The production of polyclonal antibodies is preferably performed according to the following steps: immunization of an appropriate organism like e.g. sheep with recombinantly produced N-terminal proBNP, isolation of the antibodies, screening for the most reactive epitopes and purification of the antibodies via immunosorption at suitable peptides. Such a method is described in example 2.

The production of monoclonal antibodies is preferably performed according to the following steps: immunization of a suitable organism as for example mice with recombinantly produced N-terminal proBNP and selection of the clones with regard to the reactivity of the antibodies with native N-terminal proBNP in different pools of patient sera. Such a method is described in example 3.

The invention is explained in more detail in the following examples:

### EXAMPLE 1

Method of Production of Recombinant N-Terminal proBNP (1-76)

1. Cloning of the Recombinant N-Terminal proBNP

The nucleotide sequence of the N-terminal proBNP (amino acid sequence 1-76) was produced my means of genetic synthesis. To obtain an optimum expression of the gene in E. coli the DNA sequence was suited to the codons most frequently used in E. coli. The sequences of the oligonucleotides used for the production of the gene are the following:

Pro5′ (SEQ ID NO 1):
5′CCGGATCCCACCCGCTG3′
Pro1hum (SEQ ID NO 2):.
5′CGGGATCCCACCCGCTGGGTTCCCCGGGTTCCGCTTCCGACCTGGAA
ACCTCCGGTCTGCAGGAACAGCGTAACCACCT3′
Pro2hum (SEQ ID NO 3):
5′CGGTTCCAGGGAGGTCTGTTCAACCTGCAGTTCGGACAGTTTACCCT
GCAGGTGGTTACGCTGTTCCTGC3′
Pro3hum (SEQ ID NO 4):
5′CAGACCTCCCTGGAACCGCTGCAGGAATCCCCGCGTCCGACCGGTGT
TTGGAAATCCCGTGAAGTTGCTAC 3′
Pro4hum (SEQ ID NO 5):
5′CCCAAGCTTAACGCGGAGCACGCAGGGTGTACAGAACCATTTTACGG
TGACCACGGATACCTTCGGTAGCAACTTCACGGGATTTCC3′
Pro3′ (SEQ ID NO 6):
5′CCCAAGCTTAACGCGGAGC3′

The production of the gene was carried out with these primers using PCR (polymerase chain reaction). The amplified gene was cloned in a suitable vector like for example the vector pUC19 and then sequenced. For the cloning of the gene in the expression vector pQE8 the gene was cut out of the vector pUC19 via the restriction cutting points Bam Hi and Hind III and then ligated in the vector pQE8 allowing an expression of proteins with N-terminal Histidin-Tag and transformed in E. coli M15 [pREP4].

2. Expression of the N-Terminal proBNP in E. coli

For the expression of the gene in E. coli an over-night culture of a recombinant E. coli clone was transfected 1/60 in Luria-Broth (with 100 μg/ml ampicillin and 50 μg/ml kanamycin) and induced at an OD 550 of 1 with IPTG (isopropylthiogalactoside; 1 mM final concentration). After the induction the cultures were further incubated for 4 hours at 37° C. The cultures were then centrifuged and the cell pellet gathered in 50 mM Na-phosphate buffer, pH 8.0; 360 mM NaCl. After decomposition of the cell suspension via ultrasound the suspension was centrifuged and the supernatant applied on a Ni—NTA (nitrilo-triacetate) column. After a washing step with 50 mM Na phosphate buffer, pH 8.0; 300 mM NaCl; 20 mM imidazole the histidin-tagged N-terminal proBNP was eluted with 50 mM Na-phosphate buffer, pH 8.0; 300 mM NaCl; 300 mM imidazole. The eluted fractions were gathered and dialysed against 50 mM Tris pH 8.0. To separate impurities the dialysate was applied to a Q-sepharose column. The mass of the purified N-terminal proBNP was determined via MALDI-TOF.

### EXAMPLE 2

Production of Polyclonal Antibodies Against N-Terminal proBNP

1. Immunization

Sheep were immunized with recombinant N-terminal proBNP (1-76) in complete Freund's adjuvant. The dose was 0.1 mg per animal. The immunizations were repeated at 4-week intervals in a period of 10 months. 6 weeks after the first immunization and afterwards once a month the serum samples were obtained and tested for their sensitivity and titre.

2. Purification of Polyclonal Antibodies From Sheep Serum

Starting from the raw serum of a sheep immunized with recombinant N-terminal proBNP lipid components were removed by delipidation with aerosil (1.5%). Afterwards the immunoglobulins were separated with ammonium sulphate (2M). The dissolved precipitation was dialysed against 15 mM KPO₄, 50 mM NaCl pH 7.0 and chromatographed via DEAE sepharose. The IgG fraction, PAB<rec. NT-pro-BNP>S-IgG(DE) was in the eluate.

3. Sequential Affinity Chromatography for the Production of NT-pro-BNP Specific Polyclonal Antibodies

For the purification of NT-proBNP specific polyclonal antibodies directed against the amino acids 1-21, PAB<rec. NT-pro-BNP>M-IgG (IS, 1-21) the C-terminal biotinylated peptide HPLGSPGSASDLETSGLQEQR-Bi (1-21-Bi, SEQ ID NO 7) was used. The affinity matrix was produced by the loading of 10 ml streptavidin-coated methacrylate polymer particles (Boehringer Mannheim, Ref. 1529188) with 1 mg of peptide (1-21-Bi).

With 10 ml of the affinity matrix a column was packed and equilibrated with 50 mM KPO₄, 150 mM NaCl pH 7.5 (PBS). For the first step of the sequential affinity chromatography 850 mg PAB<NT-pro-BNP>S-IgG(DE) were bound to the column. The eluate was preserved for a second step (see below). The column was washed with PBS and 20 mM KPO₄, 500 mM NaCl, 0.1% Triton X-100, 0.5% Na-deoxichole acid pH 7.5. The IgG specifically bound to the affinity matrix was eluted with ImmunoPure® Gentle Ag/Ab elution buffer (Pierce, Product N^{o} 21013). The affinity matrix was regenerated with 1M propionic acid and conserved in PBS/NaN₃.

In the same way described above the peptide Bi-ELQVEQTSL (Bi-30-38 SEQ ID NO 8) was used for the production of an affinity matrix for the purification of NT-pro-BNP-specific immunoglobulins directed against the amino acids 30 to 38. PAB<rec. NT-pro-BNP>M-IgG(IS, 30-38) was gathered from the eluate of the first affinity purification.

4. Biotinylation of PAB<NT-pro-BNP>S-IgG(IS, 1-21)

The affinity-purified antibodies are dialysed against the biotinylation buffer (100 mM KPO₄, 70 mM NaCl pH 8.0) and afterwards the solution is adjusted to a protein concentration of 1 mg/ml. D-biotinoyl-aminocaproic acid-N-hydroxysuccinimide ester is dissolved in DMSO and added to the antibody solution in a molar relationship of 1:7.5. The reaction is stopped by adding L-lysin and the surplus of the label reagent is removed by dialysis.

5. Digoxigenylation of PAB<NT-pro-BNP>S-IgG(IS, 30-38)

The affinity-purified antibodies are dialysed against the digoxigenylation buffer (100 mM KPO₄, 70 mM NaCl pH 7.6) and then the solution is adjusted to a protein concentration of 1 mg/ml. Digoxigenin-3-CME-N-hydroxysuccinimide ester is dissolved in DMSO and added to the antibody solution in a molar relationship of 1:5. The reaction is stopped by adding L-lysin and the surplus of the label reagent is removed by dialysis.

### EXAMPLE 3

Production and Screening for Monoclonal Antibodies Against N-Terminal proBNP (1-76)

1. Obtaining Monoclonal Antibodies Against NT-proBNP (1-76)

Balb/c mice, 8-12 weeks old, are subjected to intraperitoneal immunization with 100 μg recombinant N-terminal proBNP antigen, with complete Freund's adjuvant. After 6 weeks three further immunizations are performed at 4-week intervals. One week after the last immunization blood was taken and the antibody titre was determined in the serum of the test animals. From the spleen of positively reacting mice B-lymphocytes are obtained and subjected to fusion with a permanent myeloma cell line. The fusion is carried out according to the well-known method of Köhler and Millstein (Nature 256, 1975, p. 495-497). The primary cultures of hybrid cells built here are cloned in a usual way for example by using the commercially available cell sorter or by “limiting dilution”. Only those clone cultures are processed which—in a suitable test procedure—react positively with recombinant N-terminal proBNP and identify natural N-terminal proBNP in patient sera (see point 2). By this way several hybridoma cell lines producing the monoclonal antibodies according to the invention are gathered.

For the production of ascites 5×10⁶ hybridoma cells are intraperitoneally injected in Balb/c mice which had been treated 1-2 times with 0.5 ml Pristan before. After 2-3 weeks ascites liquid can be obtained from the abdominal region of the mice. From this, the antibodies can be isolated in the usual way. These monoclonal antibodies are specifically directed against human N-terminal proBNP. In the following they are called MAB M 10.1.11 or MAB M 13.4.14. Both monoclonal antibodies belong to the subclass IgG1, kappa.

By means of this method both hybridoma-cell lines clone M 10.1.11 and M 13.4.14, which were deposited with the DSMZ as mentioned above, could be isolated.

2. Screening Test for Antibodies Against proBNP Peptides and Recombinant NT-proBNP

To identify the presence and specificity of antibodies against NT-proBNP in the serum of immunized mice, in the culture supernatant of the hybrid cells or in ascite liquid the clones were evaluated according to the following test principles:

a) Reactivity with Recombinant N-Terminal proBNP

Microtitre plates (Nunc, Maxisorb) are bound with 2.5 μg/ml recombinant NT-proBNP as an antigen in a loading buffer (Boehringer, 0.2 M sodium carbonate/bicarbonate, pH 9.3-9.5, Cat. No. 726 559) 100 μl/well, for 1 hour at room temperature under stirring. The post-loading is carried out in PBS buffer-(phosphate buffered saline, Oxid, Code-BR 14a) and 1% Byco C, for 30 minutes. Subsequently, washing is performed with washing buffer (0.9 sodium chloride solution, 0.05% Tween 20). The antibody sample incubation is carried out with 100 μl/well for 1 hour at room temperature under stirring. A further washing step with washing solution takes place twice then. Afterwards, a further incubation is carried out with the detection antibody PAB<M-Fcy>S-Fab-peroxidase conjugate (Boelringer Mannheim, cat. No. 1500 686), 100 mU/ml, 100 μl/well, for 1 hour at room temperature under stirring. After a further washing step with washing buffer the peroxidase activity is established in the usual way (for example with ABTS®, for 30 minutes at room temperature, the extinction difference is read in mU at 405 nm by means of an ELISA reader.

b) Reactivity with N-Terminal proBNP Peptides:

In this case streptavidin-loaded microtitre plates are bound with NT-proBNP-peptide biotin conjugates of the sequences 1-10, 8-18, 1-21, 16-30, 30-38, 39-50, 50-63 or 64-76 as an antigen, 250 ng/ml in PBS buffer (phophate buffered saline, Oxid, Code-BR 14a) with 0.5% Byco C, 100 μl/well for 1 hour at room temperature under stirring. Subsequently, washing is carried out with washing buffer (0.9 sodium chloride solution, 0.05% Tween 20). The antibody sample incubation and the detection reaction are performed as described in a). Due to the reactivity with certain NT-proBNP peptides the position of the epitope can be determined.

c) Reactivity with Native N-Terminal proBNP in the Patient Sample

Microtitre plates (Nunc, Maxisorb) are bound with 5 μg/ml PAB<human proBNP>S-IgG (IS, (1-21) or (30-38)S-IgG in loading buffer (Boehringer, 0.2 M sodium carbonate/bicarbonate, pH 9.3-9.5, Cat. No. 728 559), 100 μl/well, for 1 hour at room temperature under stirring. The post-loading is carried out in PBS buffer (phosphate buffered saline, Oxid, Code-BR 14a) and 1% Byco C, for 30 minutes. Subsequently, washing is performed with washing buffer (0.9 sodium chloride solution, 0.05% Tween 20). The incubation with native antigen in patient plasma, diluted in PBS buffer, is carried out with 100 μl/well for 1 hour at room temperature under stirring. After a further washing step the antibody sample incubation is performed with 100 μl/well for 1 hour at room temperature under stirring. Subsequently, washing is carried out twice with washing solution and a further incubation with the detection antibody PAB<M-Fcy>S-Fab-peroxidase conjugate (Boehringer Mannheim GmbH, cat. No. 1500 686), 100 mU/ml, 100 μl/well, for 1 hour at room temperature under stirring. After a further washing step with washing buffer the peroxidase activity is established in the usual way (for example with ABTS®, for 30 minutes at room temperature, the extinction difference is read in mU at 405 nm by means of an ELISA reader).

3. Results: Reaction Pattern of the Monoclonal and Polyclonal Antibody Against N-Terminal proBNP

a) Reactivity of the MABs (c 5 μg/ml) from Immunization with N-Terminal proBNP Peptides:

TABLE 1

| | | Reactivity with | | | | | | | | | |
|---|---|---|---|---|---|---|---|---|---|---|---|
| | | proBNP peptides | | | | | | | | | Native |
| MAB | Immunogen | 1-10 | 8-18 | 1-21 | 16-30 | 30-38 | 39-50 | 50-63 | 64-76 | Rec. proBNP | proBNP |
| 5.2.27 | 1-10 | 1.42 | 0.04 | 1.48 | 0.05 | 0.03 | 0.04 | 0.04 | 0.04 | 1.16 | 0.30 |
| 2.1.4 | 16-30 | 0.04 | 0.04 | 0.04 | 1.86 | 0.04 | 0.04 | 0.04 | 0.04 | 0.1 | 0.02 |
| 1.2.6 | 39-50 | 0.04 | 0.04 | 0.03 | 0.04 | 0.03 | 1.23 | 0.03 | 0.04 | 0.44 | 0.06 |

The monoclonal antibodies obtained from immunizations with different peptides react very strongly with the corresponding peptides. The reactivity with the recombinant N-terminal proBNP can only be recognized with 2 monoclonal antibodies whereas no reaction occurs with native N-terminal proBNP in a patient pool (see table 1).

b) Reactivity of the Monoclonal Antibodies (MAB) from Immunization with Recombinant N-Terminal proBNP:

TABLE 2

| | Reactivity with | | | | | | | | | |
|---|---|---|---|---|---|---|---|---|---|---|
| | proBNP peptides | | | | | | | | | Native |
| MAB | 1-10 | 8-18 | 1-21 | 16-30 | 30-38 | 39-50 | 50-63 | 64-76 | Rec. proBNP | proBNP |
| 10.1.11 | 0.04 | 0.97 | 1.03 | 0.04 | 0.04 | 0.06 | 0.04 | 0.04 | 1.61 | 1.70 |
| 10.3.19 | 0.04 | 0.04 | 0.04 | 0.04 | 0.04 | 0.05 | 0.04 | 0.03 | 1.24 | 0.91 |
| 10.3.30 | 0.04 | 0.04 | 0.03 | 0.04 | 0.04 | 0.06 | 0.04 | 0.03 | 1.43 | 0.79 |
| 13.4.14 | 0.04 | 0.04 | 0.04 | 0.04 | 0.04 | 0.05 | 0.03 | 0.04 | 1.65 | 1.83 |
| 13.1.18 | 0.04 | 0.04 | 0.03 | 0.04 | 1.14 | 0.03 | 0.04 | 0.04 | 1.47 | 0.56 |
| 13.2.22 | 0.04 | 0.04 | 0.04 | 0.04 | 0.04 | 0.03 | 0.04 | 0.04 | 1.82 | 1.61 |

The monoclonal antibodies obtained from immunization with recombinant N-terminal proBNP only react partially with peptides, but very strongly with the recombinant N-terminal proBNP or native N-terminal proBNP in a patient pool. The non-reaction of single monoclonal antibodies with the peptides points to the identification of the so-called conformation epitopes (see table 2).

c) Reactivity of the PABs From Immunization with Recombinant N-Terminal proBNP:

TABLE 3

| | | Reactivity with | | | | | | | | | |
|---|---|---|---|---|---|---|---|---|---|---|---|
| | | proBNP-peptides | | | | | | | | | Native |
| MAB | Immunosorption | 1-10 | 8-18 | 1-21 | 16-30 | 30-38 | 39-50 | 50-63 | 64-76 | Rec. proBNP | proBNP |
| S-9212 | Without | 0.13 | 1.81 | 1.98 | 1.16 | 2.99 | 0.83 | 1.22 | 0.06 | 0.89 | — |
| S-9212 | 1-21 | 0.99 | 2.99 | 2.99 | 1.00 | 0.20 | 0.13 | 0.20 | 0.15 | 1.98 | 1.41 |
| S-9212 | 30-38 | 0.08 | 0.07 | 0.07 | 0.07 | 2.99 | 0.06 | 0.17 | 0.06 | 2.99 | 1.41 |

The PAB obtained showed the strongest reaction with the peptides 1-21 and 30-38. For this reason these epitopes were chosen and the PAB was positively immunosorbed with the aid of these peptides. The PAB immunosorbed with peptide 1-21 shows the strongest reaction with the region 8-20 and a clearly reduced reaction with the N-terminal sequence 1-10. The PABs immunosorbed this way react very strongly with the recombinant N-terminal proBNP and in the PAB/PAB sandwich format with the native sample (see table 3).

### EXAMPLE 4

Highly Sensitive Immunoassay for the Determination of NT-proBNP

Using the antibodies produced in example 2 and 3 a highly sensitive immunoassay could be built. In general, all test formats applying 2 antibodies with a different epitope recognition are suitable. As an example the so-called sandwich-ELISA is described.

As a solid phase a microtitre plate (MTP) coated with streptavidin was used. 10 μl of an untreated sample or calibrator is pipetted together with 100 μl of buffer containing both epitope-specific antibodies into the MTP cups and then incubated for one hour at room temperature. As an antibody 1 μg/ml of biotinylated PAB<rec.NT-proBNP>S-IgG(IS, 1-21) and 0.5 μg/ml of digoxigenylated PAB<rec.NT-proBNP>S-IgG(IS, 30-38) were used. Afterwards the solution is sucked off and washed three times with 350 μl washing buffer. Then 100 μl of the conjugate solution are added by a pipette and incubated again for 1 h at room temperature. As a conjugate an anti-digoxin-antibody-POD conjugate with a concentration of 100 mIU/ml is used. The conjugate solution is then sucked off and washed 3 times with 350 μl of washing buffer. At the end ABTS® substrate solution is pipetted into the wells and measured for 30 minutes at room temperature. After reaching the substrate reaction of 30 minutes the microtitre plate is directly measured in an MTP reader at a wave length of 405 nm and against the reference wave length of 495 nm.

To determine the sensitivity a calibration curve was established and the precision of the zero standard (n=21) was determined. As calibrators human EDTA plasma was used which was then built up with the recombinant N-terminal proBNP in the concentration required. Bovine plasma was used as the zero standard. The results are shown in table 4.

TABLE 4

| | | Standard deviation |
|---|---|---|
| | Extinction (mean) | (n = 21) |
| Calibrator a: 0 fmol/ml | 131 mU | 5.7 mU |
| Calibrator b: 5.04 fmol/ml | 268 mU | |
| Calibrator c: 19.9 fmol/ml | 746 mU | |
| Calibrator d: 50.5 fmol/ml | 1500 mU | |
| Calibrator e: 100.9 fmol/ml | 2401 mU | |

On the basis of the calibration curve gradient of 22.5 mU×ml/fmol and an SD of 5.7 mU the following lower detection limit is given according to the Kaiser formula:
LDL=3 SD zero standard/Ccgradient=3×5.7/22.5=0.76 fmol/ml.

### EXAMPLE 5

Determination of the Sample Stability of N-Terminal proBNP

With the help of the sandwich ELISA described in example 4 the analyte stability of N-terminal proBNP was measured. For this blood was taken from 4 patients of the NYHA-class II-III into EDTA-containing collector tubes and preserved at room temperature for 3 days. Each day a sample was taken and the concentration of N-terminal proBNP was measured. The reference sample as well as the samples for the determination of the stability in EDTA plasma were directly cooled down to 4° C.-8° C. and centrifuged within 15 minutes. The EDTA plasmas were preserved at 4° C. and at room temperature and then measured at different times within a 24-hour stress duration. The results are depicted in table 5.

TABLE 5

| Stress time | | Recovery (%) |
|---|---|---|
| EDTA-whole blood, room temp. | 24 h | 98.8 |
| | 48 h | 98.0 |
| | 72 h | 100.5 |
| EDTA-plasma, 4° C. | 2 h | 97.5 |
| | 4 h | 98.5 |
| | 6 h | 102.0 |
| | 24 h | 103.0 |
| EDTA-plasma, room temperature | 2 h | 103.0 |
| | 4 h | 104.8 |
| | 6 h | 102.0 |
| | 24 h | 96.0 |

These data prove that N-terminal proBNP is completely stable within the times tested and can therefore be used as a routine parameter. This result is inconsistent with the literature (Hunt et. Al., Clinical Endocrinology, 47, 287 (1997)) and confirms the assumption that by the selection and design of this test format with 2 specific antibodies the epitopes of which are not at the external end of the analyte the analyte stability can be influenced.

### EXAMPLE 6

Determination of the Diagnostic Sensitivity of the N-Terminal proBNP Assay

For the determination of the diagnostic sensitivity the test described in example 4 was used again. For this, 114 healthy individuals and 235 patients of the NYHA-classification between 1 and IV were measured. Normally it is particularly critical to differentiate healthy individuals from patients of NYHA class I.

With this highly-sensitive assay a median value of 6.6 fmol/ml NT-proBNP with a standard deviation of 7.3 fmol/ml was measured in 110 healthy blood donors. The lowest level measured was 0.2 fmol/ml. This shows clearly that a sensitivity of <1.0 fmol/ml is necessary to exactly detect the reference region. With this distribution the upper normal value region (97.5% percentile) determined was 26.6 fmol/ml.

Assuming a reference region of 0-26.6 fmol/ml only 16 patients out of 233 patients of the NYHA classification I-IV showed a value in the standard region. This corresponds to a clinical sensitivity of 93.3%. If only patients with the NYHA classification I are considered 30 out of 37 patients are detected as positive which corresponds to a sensitivity of 81.1%.

This result confirms that by the highly sensitive N-terminal proBNP assay a clear differentiation between patients suffering from NYHA class I heart failure and a healthy normal collective is possible. With the state of the art assays (Dagubatti et al., Cardiovascular Research 36 (1997), 246) available until now this could not be achieved.

1-19. (canceled)

20. A method of identifying N-terminal proBNP comprising:
detecting a complex of a first antibody, a second antibody, and N-terminal proBNP in a urine sample; wherein
the first antibody is specific to a first epitope of the N-terminal proBNP;
the second antibody is specific to a second epitope of the N-terminal proBNP; and
the first epitope and the second epitope are different.

21. The invention of claim 20, wherein at least one of the first and the second antibodies comprises a label, and wherein the method further comprises detecting a signal emitted from the label.

22. The invention of claim 20 wherein the first and the second antibodies bind simultaneously to the N-terminal proBNP.

23. The invention of claim 20 wherein the detecting is performed by a heterogeneous test procedure.

24. The invention of claim 23 wherein the test procedure comprises a sandwich assay.

25. The invention of claim 21 wherein the first antibody is labelled with biotin.

26. The invention of claim 25 wherein the second antibody is labelled with ruthenium.

27. The invention of claim 26 wherein the detecting comprises an electrochemoluminescence assay.

28. A method of differentiating a sample taken from a healthy patient and a sample taken from a patient with a type of heart failure, comprising:
identifying an amount of N-terminal proBNP in the urine sample with the method of claim 20; and
correlating the amount of N-terminal proBNP identified in the urine sample with a level of N-terminal proBNP characteristic of a healthy patient or a patient with a type of heart failure;
wherein the type of heart failure is selected from the group consisting of NYHA Class I, NYHA Class II, NYHA Class III, and NYHA Class IV.

29. The invention of claim 28 wherein the type of heart failure is NYHA Class I.

30. The invention of claim 20 further comprising quantifying the N-terminal proBNP identified in the urine sample against a standard comprised of recombinant N-terminal proBNP.

31. A method of identifying N-terminal proBNP comprising:
binding a first antibody to N-terminal proBNP in a urine sample;
binding a second antibody to the N-terminal proBNP; and
detecting a complex of the N-terminal proBNP, the first antibody, and the second antibody; wherein
the first antibody is specific to a first epitope of the N-terminal proBNP;
the second antibody is specific to a second epitope of the N-terminal proBNP;
the first antibody and the second antibody bind simultaneously to the N-terminal proBNP; and
the first epitope and the second epitope are different.

32. The invention of claim 31, wherein at least one of the first and the second antibodies comprises a label, and wherein the method further comprises detecting a signal emitted from the label.

33. The invention of claim 31 wherein the detecting is performed by a heterogeneous test procedure.

34. The invention of claim 33 wherein the test procedure comprises a sandwich assay.

35. The invention of claim 32 wherein the first antibody is labelled with biotin.

36. The invention of claim 35 wherein the second antibody is labelled with ruthenium.

37. The invention of claim 36 wherein the detecting comprises an electrochemoluminescence assay.

38. A method of differentiating a sample taken from a healthy patient and a sample taken from a patient with a type of heart failure, comprising:
identifying an amount of N-terminal proBNP in the urine sample with the method of claim 31; and
correlating the amount of N-terminal proBNP identified in the urine sample with a level of N-terminal proBNP characteristic of a healthy patient or a patient with a type of heart failure;
wherein the type of heart failure is selected from the group consisting of NYHA Class I, NYHA Class II, NYHA Class III, and NYHA Class IV.

39. The invention of claim 38 wherein the type of heart failure is NYHA Class I.


SEQUENCE LISTING (8 sequences)

SEQ ID NO: 1 - DNA, 17 bp, Escherichia coli
ccggatcccacccgctg

SEQ ID NO: 2 - DNA, 79 bp, Escherichia coli
cgggatcccacccgctgggttccccgggttccgcttccgacctggaaacctccggtctgcaggaacagcgtaaccacct

SEQ ID NO: 3 - DNA, 70 bp, Escherichia coli
cggttccagggaggtctgttcaacctgcagttcggacagtttaccctgcaggtggttacgctgttcctgc

SEQ ID NO: 4 - DNA, 71 bp, Escherichia coli
cagacctccctggaaccgctgcaggaatccccgcgtccgaccggtgtttggaaatcccgtgaagttgctac

SEQ ID NO: 5 - DNA, 87 bp, Escherichia coli
cccaagcttaacgcggagcacgcagggtgtacagaaccattttacggtgaccacggataccttcggtagcaacttcacgggatttcc

SEQ ID NO: 6 - DNA, 19 bp, Escherichia coli
cccaagcttaacgcggagc

SEQ ID NO: 7 - protein, 21 aa, Escherichia coli
HPLGSPGSASDLETSGLQEQR

SEQ ID NO: 8 - protein, 9 aa, Escherichia coli
ELQVEQTSL